(19) United States
(12) Patent Application Publication
(10) Pub. No.: US 2003/0091558 A1
(43) Pub. Date: May 15, 2003

(54) Storage-stable fibrinogen solutions

(76) Inventor: Christopher J. Woolverton, Kent, OH (US)

Correspondence Address: Evelyn H. McConathy, Esquire, Dilworth Paxson LLP, 3200 Mellon Bank Center - 1735 Market Street, Philadelphia, PA, 19103, US

(21) Appl. No.: 10/263,987

(22) Filed: Oct. 3, 2002

Related U.S. Application Data
(60) Provisional application No. 60/326,963, filed on Oct. 3, 2001.

(51) Int. Cl. A61K 38/48

(52) U.S. Cl. 424/94.64

(57) ABSTRACT

Methods are provided for the stable storage of ready-to-use, biocompatible mammalian fibrinogen, which despite its concentration, remains available in fluid form, and which will permit long-term rapid and easy processing into a tissue adhesive preparation. Also provided is the sterile, storage-stable aqueous fibrinogen product resulting from the use of the present methods, wherein the fibrinogen remains long term in ready-to-use in liquid form, it has not spontaneously clotted (i.e., formed a clot even in the absence of an activator, such as thrombin/Ca⁺⁺), and it retains its biological activity (i.e., the ability to rapidly form a fibrin clot upon exposure and vigorous mixing with thrombin and Ca⁺⁺).

### REFERENCE TO RELATED APPLICATIONS

This application claims priority to U.S. Provisional Application No. 60/326,963, filed Oct. 3, 2001, herein incorporated in its entirety.

### FIELD OF THE INVENTION

This invention relates generally to storage-stable, concentrated fibrinogen preparations and a method of use therefor to prevent blood loss, to promote wound healing, and for many other therapeutic and non-therapeutic applications.

### BACKGROUND OF THE INVENTION

Fibrinogen is a blood plasma protein, serving a significant role in the final stage of the coagulation to preserve hemostasis and prevent blood loss in mammals. Clot formation in mammals, i.e., blood coagulation, occurs by means of a complex cascade of events in which in the final steps the monomeric form of fibrinogen reacts with thrombin and activated Factor XIII in the presence of calcium ions, to form a fibrin clot comprising a cross-linked fibrin polymer.

The fibrinogen monomer, representing 2-4 grams/liter of blood plasma protein, consists of three pairs of disulfide-linked polypeptide chains. These are designated (Aα)₂, (Bβ)₂, representing the two small aminoterminal peptides of the α and β chains, respectively), and γ₂. Cleavage of the fibrinopeptide A from fibrinogen by thrombin results in the compound, fibrin I, and the subsequent cleavage of fibrinopeptide B results in the final fibrin II compound. The cleavage only slightly reduces the molecular weight of fibrinogen from 340,000 daltons to only 334,000, but the process exposes the essential polymerization sites to permit formation of the assembled and cross-linked fibrin clot. See, Jackson, Ann. Rev. Biochem 49:765-811 (1980); Furie et al., Cell 53:505-518 (1988).

Recently, biological adhesives have been developed comprising fibrinogen, thrombin and other components, which imitate the final stages of natural coagulation, thereby resulting in a fibrin clot. Called fibrin- or tissue-sealant, biological sealant, fibrin- or tissue-glue, biological adhesive, or the like (collectively referred to herein as a “fibrin sealant”), tests on such materials have shown a direct relationship between tensile strength and the final fibrinogen concentration (Japanese Patent Unexamined Published Application, Kokai No. Sho 61-293443). Thus, the availability of concentrated fibrinogen is significant for the preparation of conventional fibrin sealants.

Tissue adhesives based on fibrinogen are known, for example from U.S. Pat. No. 6,117,425 (MacPhee et al.) In addition to fibrinogen and Factor XIII, such formulations may also contain additional proteins, such as fibronectin and albumin, and optionally antibiotic agents, growth factors, and the like. The required catalytic (thrombin-mediated) activity can either originate from the host tissue (the wound surface) to which it is applied, or it can be added in the form of a thrombin and Ca⁺⁺ ion-containing solution or powder to the tissue adhesive in the course of application. Such fibrin sealants have been used for seamless and/or seam-supporting binding of human or animal tissue or organ parts, for wound sealing, hemostasis and promoting wound healing, for coating prosthetic devices, and for many other therapeutic and non-therapeutic applications.

The fibrinogen component of fibrin sealants is derived from pooled blood plasma, often as a waste product in the preparation of Factor VIII. Fibrinogen can be concentrated from plasma by cryoprecipitation, or by precipitation by known methods using various reagents, e.g., polyethylene glycol, ether, ethanol, ammonium sulfate or glycine. Fibrin sealants are reviewed, for example, by Brennan, Blood Reviews 5:240-244 (1991); Gibble et al., Transfusion 30:741-747 (1990); Matras, J. Oral Maxillofac. Surg. 43:605-611 (1985); Lerner et al., J. Surg. Res. 48:165-181 (1990).

From the standpoint of preparation, according to U.S. Pat. No. 5,290,552, early surgical adhesive formulations necessarily contained a high fibrinogen content (about 8-10%), from which lyophilates were extremely difficult to prepare. Such cryoprecipitates were relatively unstable, and required storage below −20° C. until use. Formulations to improve the stability of the cryoprecipitate included adding inhibitors of plasminogen activators or albumin.

At a sufficiently high fibrinogen concentration, the preparations provide effective hemostasis, good adherence of the seal to the wound and/or tissue areas, high strength of the adhesions and/or wound sealings, and complete resorbability of the adhesive in the course of the wound healing process. For optimal adhesion, a concentration of fibrinogen of about 15 to 60 mg/ml of the ready-to-use tissue adhesive solution is required (MacPhee, personal communication, 1995).

Tissue adhesives are marketed either in the form of deep-frozen solutions or as a lyophilate. This is because as a liquid solution, highly concentrated fibrinogen is known to be highly unstable (http:www.tissuesealing.com/us/products/biological/monograph. cfm), i.e., it is subject to spontaneous coagulation. Consequently, commercially available lyophilized and/or deep-frozen fibrinogen concentrates, such as Tissucol, must currently be liquefied, i.e., slowly thawed (“melted”) or reconstituted from lyophilized form before application. Both liquefaction processes, however, are associated with significant effort and a considerable time lag before the product can be used, which can place an already injured patient into a life-threatening situation.

The “liquefaction temperature” of the deep-frozen concentrate, e.g., the point at which the preparation changes from frozen solid to liquid, requires slowly increasing the temperature of the solution—generally to at least 25° C., more often to over 37° C., with significant stirring or agitation for up to 30-60 minutes (http://www.tissuesealing.com/us/products/biological/monograph.cfm). As a result, reconstitution of prior art fibrinogen preparations requires the use of a water bath or other heating device (typically at 37° C.) to convert the deep-frozen material to a ready-to-use solution in the shortest possible time. However, heat exchange is typically made even more difficult because of the necessary double coating packaging required, for example to maintain sterile conditions of the product, throughout the difficult and cumbersome thawing procedure. For instance, deep-frozen fibrin sealant preparations in pre-filled, ready-to-use, sterile disposable syringes must be double scaled in plastic film for reasons of sterility.

The transition from deep-frozen solid to liquid state does not occur abruptly, but over a progression of increasing temperature steps, passing through gelatinous and viscous transitory states. According to at least one test, a sample is not designated a ‘liquid’ until a horizontal liquid level forms when tipping the test tube, i.e., when the sample does not form a visible bulge immediately upon flowing. Thus, testing the product to determine when it has uniformly reached the ‘liquid’ ready-to-use state adds additional time-consuming steps before the stored prior art fibrinogen preparations can be used. Furthermore, a degree of uncertainty and potential for error by the practitioner is apparent that can affect the utility and effectiveness of the fibrinogen product.

The preparation time of lyophilized fibrinogen also results in significant delays before the product can be used, which becomes a real problem in the use of currently available fibrinogen-based hemostats. Therefore, significant effort has been undertaken to improve the solubility of lyophilized fibrinogen preparations. For example, one manufacturer requires the use of a magnetic stirrer added to the vials of protein to provide significant agitation while heating. This results in dissolution times which are faster than those obtained for the same product without significant mixing, but it still requires 30-60 minutes of preparation time simply to get the fibrinogen ready to use.

The solubility of fibrinogen preparations of the prior art is often further reduced by the implementation of virus inactivation methods. These are preferably carried out in a manner such that the lyophilized material is subjected to a heat treatment, for example according to EP 0 159 311.

It is known that the reconstitution of lyophilates can be improved by the addition of certain additives. Thus, for example, EP-0 345 246 describes a lyophilized fibrinogen preparation which, in addition to fibrinogen, further contains at least one biologically acceptable additive (a tenside). The addition of tensides results in an improved wetting of the lyophilisate with the solvent, whereby the rate of dissolution at a certain temperature is improved, but not the solubility of the fibrinogen itself. Therefore, such preparations must also be reconstituted in a surrounding temperature over 25° C., usually 37° C.

To overcome the need to reconstitute or liquefy lyophilized or deep-frozen fibrinogen products before use, especially concentrated preparations, certain fibrinogen preparations have been introduced which are soluble at room temperature. However, such prior art products are cytotoxic (Beriplast, Biocol, Bolheal HG-4).

U.S. Pat. No. 5,962,405 provides storage-stable lyophilized or deep frozen liquid preparations of fibrinogen, which can be reconstituted and liquefied into ready-to-use fibrinogen and/or tissue adhesive solutions—preferably without the use of additional means, such as heating and/or stirring devices, to produce ready-to-use tissue adhesive solutions having a fibrinogen concentration of at least 70 mg/ml. However, the preparations comprise fibrinogen and at least one additional substance which improves the solubility of the preparations, and/or lowers its liquefaction temperature, and reduces the viscosity of a ready-to-use tissue adhesive solution at room temperature. The solubility enhancing substance, selected from one or more of the following substances: benzene, pyridine, piperidine, pyrimidine, morpholine, pyrrole, imidazole, pyrazole, furan, thiazole, purine compounds or vitamins, nucleic bases, nucleosides or nucleotides, is added at a rate of 0.03-1.4 mmol per gram fibrinogen, although the relatively higher ratios of substance/fibrinogen are recommended. Additional proteins, adjuvants and additives may also be present. However, because the liquefaction temperature is lowered, the '405 patent claims that liquefaction of the deep-frozen, concentrated fibrinogen solution is advantageously possible in a surrounding temperature of 20°-23° C. (room temperature), as opposed to the previously required 37° C. warming conditions. Nevertheless, the method still requires storage under deep-frozen conditions (temperatures maintained at −25° C. to below −15° C.), and the preparations still take up to 15 minutes to liquefy.

An alternative solution to the premature coagulation of the fibrinogen solution for use in tissue sealant preparations, U.S. Pat. No. 5,985,315 provides a stable biological pre-activated adhesive comprising fibrinogen with the addition of at least one activated coagulation factor whose activation does not depend on calcium ions. The preactivated adhesive is stable in aqueous solution, i.e., the solution does not coagulate spontaneously for at least one hour at a temperature of 20°; but it can be made to coagulate about 5 minutes simply by adding calcium ions. No additional activator is required. Thus, the resulting biological adhesive requires neither the addition of thrombin or prothrombin to achieve coagulation. Unfortunately, however, such a slow coagulation time would make the use of the resulting fibrin sealant impractical for use on any type of a flowing or pulsating wound.

From a medical standpoint, therefore, the quick availability of ready-to-use, biological, tissue adhesives is essential, especially in surgical emergency situations. Additionally, as little manipulation as possible should be required for the preparation of the ready-to-use fibrin sealant solution to minimize the burden on the assisting personnel. Fibrin sealant preparations require a stored fibrinogen component, but at the present time the fibrinogen is only available as a lyophilate, a deep-frozen concentrate, or as a mixture with other components that could negatively alter the effectiveness of the fibrinogen-based tissue adhesive or its safe use with a patient or subject. Thus, there remains a need for a storage-stable, ready-to-use fibrinogen solution, which despite its high concentration, remains available in fluid form, and which will permit rapid and easy processing into a tissue adhesive preparation.

### SUMMARY OF THE INVENTION

The present invention comprises methods for the stable storage of ready-to-use, biocompatible mammalian fibrinogen, which despite its concentration, remains available in fluid form, and which will permit rapid and easy processing into a tissue adhesive preparation. Also provided is the sterile, storage-stable aqueous fibrinogen product resulting from the use of the present methods, wherein the fibrinogen remains ready-to-use in liquid form, it has not spontaneously clotted (i.e., formed a clot even in the absence of an activator, such as thrombin/Ca⁺⁺), and it retains its biological activity (i.e., the ability to rapidly form a fibrin clot upon exposure and vigorous mixing with thrombin and Ca⁺⁺). The subject stored concentrated, ready-to-use, biocompatible mammalian fibrinogen is fully solubilized, the solution is aqueous, and its stability is pH and temperature dependent. The product can be frozen, thawed, refrozen and re-thawed without affecting the clotting properties of the composition. The exemplified mammalian fibrinogen is bovine, but the invention need not be so limited and is directed to any mammalian fibrinogen.

The methods of the invention provide a stable, concentrated, ready-to-use, biocompatible mammalian fibrinogen solution, wherein stability is maintained for a storage period ranging from at least one (1) day to one or more years following initial preparation.

In accordance with a preferred method, the invention provides a ready-to-use fibrinogen solution, which is freshly prepared, or freshly isolated and purified from plasma, or frozen preparations of either one, and maintained under sterile conditions in a suitable container at room temperature or under refrigeration (about 4° C.), at pH levels ranging from pH 6.5 to 8.2. Stability is maintained for at least one year or more. Further provided is the ready-to-use, sterile, stable aqueous fibrinogen solution stored in accordance with the present method.

In accordance with yet other preferred methods, the invention provides for the addition of protease inhibitor(s) to the above-described ready-to-use fibrinogen solutions to enhance their storage stability. Accordingly, the invention provides a method of stably storing mammalian fibrinogen in a ready-to-use, aqueous solution, comprising freshly preparing a fibrinogen solution, or freshly isolating and purifying a fibrinogen solution from plasma under sterile conditions; adding to the fibrinogen solution an effective amount of a protease inhibitor to prevent proteolysis of the fibrinogen sample; and storing the fibrinogen solution at (i) a constant temperature ranging from about 4° C. to about 23° C., wherein the fibrinogen solution remains liquid; (ii) at pH levels ranging from pH 6.31 to 8.1, (iii) under conditions wherein biocompatibility and biological activity of the fibrinogen is maintained. Stability is maintained for at least one year or more. Further provided is the ready-to-use, sterile, stable aqueous fibrinogen solution stored in accordance with the present method.

Other additives or components are in certain embodiments also added to the above-described, storage stable, ready-to-use fibrinogen solutions to enhance the effectiveness of the resulting fibrinogen in later applications, or in products or materials produced therefrom. Further provided is the ready-to-use, sterile, stable aqueous fibrinogen solution stored in accordance with such alternative methods.

The thus-prepared and stored, ready-to-use, concentrated mammalian fibrinogen solutions may be neutralized and used without additional steps or processes in the preparation of biological tissue adhesives or sealants, including instant fibrin sealant preparations, and for other pharmacologic or cosmetic uses involving, e.g., wound healing, coagulation, fibrinogenaemia, inhibition of operative or post-operative sequelae, coating vascular prostheses, or infusion purposes, as well as for other supplemented or unsupplemented therapeutic or non-therapeutic applications in vivo or in vitro.

Additional objects, advantages and novel features of the invention will be set forth in part in the description, examples and figures which follow, and in part will become apparent to those skilled in the art on examination of the following, or may be learned by practice of the invention.

### DESCRIPTION OF THE DRAWINGS

The foregoing summary, as well as the following detailed description of the invention, will be better understood when read in conjunction with the appended drawings. For the purpose of illustrating the invention, there are shown in the drawings, certain embodiment(s) which are presently preferred. It should be understood, however, that the invention is not limited to the precise arrangements and instrumentalities shown.

FIGS. 1A and 1B are photographs of a non-reduced (FIG. 1A) and reduced SDS PAGE (FIG. 1B) of bovine fibrinogen samples after 44 days of storage at room temperature. The lanes are identical in each of the two gels. 1=MW standard; 2=bovine fibrinogen control; 3=sample buffered with pH 7.24 histidine; 4=sample buffered with pH 9.31 glycine; 5=sample buffered with pH 9.05 carbonate; 6=sample buffered with pH 9.86 carbonate; 7=bovine fibrinogen control.

### DESCRIPTION OF PREFERRED EMBODIMENTS OF THE INVENTION

The invention provides methods for the stable storage of ready-to-use fibrinogen, which despite its concentration, remains available in fluid form, and which will permit rapid and easy processing into a tissue adhesive preparation. Also provided is the storage-stable, aqueous fibrinogen product resulting from the use of the present methods.

The ready-to-use, aqueous fibrinogen solution of the present invention is “storage-stable” when after a period of days it remains stable in liquid form, it has not spontaneously clotted (i.e., formed a clot even in the absence of an activator, such as thrombin/Ca⁺⁺), and it retains its biological activity (i.e., the ability to rapidly form a fibrin clot upon exposure and vigorous mixing with thrombin and Ca⁺⁺). The disclosed methods set forth conditions under which fibrinogen is stored in a ready-to-use, aqueous solution for a substantial period of time and remains active and stable (storage-stable).

As used herein “activity” with regard to the storage-stable fibrinogen solution refers to “biological activity” of the protein, and “biological activity” refers to the one or more activities known to be associated with fibrinogen, such as the ability to rapidly form a fibrin clot as described above, or a subset thereof, in vitro and/or in vivo. Methods to assess biological activity are known to those in the art.

In the present disclosure, unless defined otherwise, all technical and scientific terms used herein have the same meaning as is commonly understood by one of ordinary skill in the art to which the invention pertains.

The storage method of the present invention is applied to any fibrinogen preparation, whether isolated and purified from blood plasma, or recombinantly prepared, or whether freshly isolated, or freshly prepared from a lyophilized or deep-frozen preparation. The methods of the present invention are applicable regardless of the length of time the fibrinogen preparation has been lyophilized or deep-frozen, so long as the biological activity of the freshly prepared fibrinogen solution is equivalent to a comparable sample of isolated and purified fibrinogen from plasma, and spontaneous clotting has not been induced in the solution.

The preferred embodiments of the invention are applicable to a crude fibrinogen product in the course of preparation, or to a final, concentrated fibrinogen preparation having greater than 90% protein purity and being greater than 95% clottable protein, or to any concentration of fibrinogen there between. For instance, in the Examples that follow, the bovine fibrinogen preparation had 61% protein purity and 97% clottable protein, while in other examples conducted by the inventors using human fibrinogen (data not shown), the preparation had 53% protein purity and 95% clottable protein. Nevertheless, the methods of the present invention were applicable to both.

In a preferred and representative embodiment of the invention the methods of storage are applied to a concentrated bovine fibrinogen preparation. The storage-stable fibrinogen preparations of the present invention, although highly concentrated, remain solubilized in aqueous solution making the fibrinogen particularly suitable for use in the preparation of supplemented or unsupplemented, ready-to-use biological tissue adhesives. The fibrinogen is optimally stored at a concentration of 10-85 mg/ml, more preferably at a concentration of 15-75 mg/ml, even more preferably at a concentration of 30-70 mg/ml, and most preferably at a concentration of 40-65 mg/ml when is used to prepare a ready-to-use tissue adhesive preparation.

Moreover, the concentration of fibrinogen, or fibrinogen-containing protein, in the storage-stable aqueous solution of the present invention generally ranges from 2 to 10 w/v %, preferably 4-7 w/v %. The concentration of fibrinogen is determined by protein absorbance measurements at 280 nm (using 14 as the extinction of 1% fibrinogen solution).

The storage-stable fibrinogen of the present invention is fully solubilized in an aqueous solution, that is, in a water-based solution. Optimal temperature and pH of the preparation would be known in accordance with the present invention, or both could be rapidly determined, by one of ordinary skill in the art using known means. However, aqueous-based gels could also be used in the present invention, so long as such material permits the complete solubilization of the fibrinogen contained therein, and so long as the preparation is sufficiently fluid as to permit the rapid preparation of ready-to-use biological tissue adhesives or other applications following storage in accordance with the methods disclosed herein. A key to the present invention is the fact that the fibrinogen solution is stably stored in ready-to-use fluid form; it is neither stored as a lyophilized preparation, nor is it in a deep frozen state.

In a preferred embodiment of the invention, fresh fibrinogen solutions are free flowing liquids that readily move along an inverted test tube, although their viscosity is typically greater than water. Stored samples that are biologically active (i.e., clot in the presence of thrombin and Ca ions) have essentially the same physical characteristics as fresh samples. This type of clotting produces the controlled clot formed using active fibrinogen when tissue adhesives are prepared and used. For the purposes of discussion, this type of clot is referred to herein simply as a “fibrin clot” to differentiate the process from a “spontaneous clot,” wherein the latter may occur in an unstable, concentrated fibrinogen solution, even absent thrombin or another activator.

However, the terms are used herein only for the purpose of distinguishing the desired uses of the stored fibrinogen solutions in which the activity of the stably stored fibrinogen solution is quickly demonstrated by the rapid formation of a fibrin clot when equal amounts of the fibrinogen and thrombin/Ca⁺⁺ are vigorously mixed, from a spontaneous clot which is indicative of instability in the prior art fibrinogen solutions. The fact that prior art, aqueous solutions of freshly-prepared fibrinogen are known to be highly unstable, and tend to spontaneously clot upon storage, makes the storage of fibrinogen in ready-to-use liquid form impractical for even a day or two using previously recognized methods.

Spontaneous clotting is recognized as an increase in viscosity (without exposure to an activator, e.g., thrombin and Ca ions), resulting in visibly decreased movement (flow) upon mixing. Often spontaneous clotting occurs in prior art, freshly-prepared, aqueous fibrinogen solutions in less than 1 day, often in only a few hours or less. The process is irreversible, leaving the fibrinogen useless for uses such as the preparation of a fibrin sealant. The instability makes the length of time that the fibrinogen could be stored in ready-to-use form using current methods completely unpredictable, and hence, unreliable.

In a preferred embodiment of the invention, the storage-stable fibrinogen is stored in a polymer, plastic or plastic-based container, although more preferably the plastic container is polypropylene. Glass is not to be used to store fibrinogen or platelets because glass enhances spontaneous clot formation.

Stored solutions of ready-to-use fibrinogen that do not clot with added thrombin and calcium ions and remain fluid (having viscosities similar to water) are referred to as “thrombin-insensitive.” However, analysis of such thrombin insensitive fibrinogen samples by SDS-PAGE (sodium dodecylsulfate polyacryamide gel electrophoresis) has shown that the fibrinogen protein has been irreversibly degraded to small molecular weight fragments. Thus, the preparation no longer contains active fibrinogen, and is not the subject of the present invention.

After addition of thrombin/Ca⁺⁺ to the ready-to-use fibrinogen solution, the rapid increase in viscosity and decrease in liquid movement that is seen, is referred to as a “gel.” In the gel state, the fibrinogen solution no longer flows freely, but can be forced to move with agitation. Although this measurement is subjective, the estimated variability is only ±2 seconds.

“Clot” formation is the sudden solidification of the fibrinogen solution, beyond which agitation cannot force liquid to flow from the solidified material. The immobile material usually becomes macroscopically opaque white and viscoplastic. Scanning electron micrographs (SEM) photographs of typical physiological or non-physiological fibrin clots are shown, for example, in Redl et al., Medizinische Welt 36:769-76 (1985). The clot generally adheres to the test tube wall and cannot be dislodged by sharp tapping of the tube on a solid surface. This measurement is less subjective than gel formation, and estimated uncertainty is only ±1 second for rapidly setting samples (8-12 seconds), although it may be slightly greater for slower clotting (>100 seconds) samples.

The temperature of the solution during storage is not particularly restricted, so long as the fibrinogen contained therein remains stable (i.e., neither inactivated nor spontaneously clotted). The preferred temperature for storage of the fibrinogen solutions of the present invention ranges from 1° to 25° C., more preferably from about 4° to about 23° C. When refrigerated, the optimal temperature is about 4° C. ±1° C. When storage is at room temperature, the optimal temperature ranges from about 20° to 25° C., more preferably from about 22° to 24° C., most preferably the temperature is about 23° C.±1° C.

To assess the effect of clot formation after freezing, samples of fibrinogen solutions were also frozen and thawed prior to testing, and it was determined that one or more freeze/thaw cycles do not appear to negatively effect the clotting ability of mammalian fibrinogen solutions even after five months storage at 4° C. prior to freezing. Together, these data strongly suggest that a liquid fibrinogen product can be readily formulated to provide at least one year of shelf life, with additional years of shelf life possible if the liquid fibrinogen is initially frozen.

The pH value of the aqueous fibrinogen solution is preferably adjusted during storage to approximately pH 5 to 8, more preferably pH 6.2-7.5. The optimal pH for the storage of a particular fibrinogen solution depends in part upon the temperature at which the material is to be stored, as is shown in the Tables that accompany the Examples which follow. However, in light of the information provided herein, one of ordinary skill in the art would be able to select the optimal pH for the fibrinogen solution based upon the planned storage temperature and conditions, knowing that the determining factor is whether the protein contained therein remains stable (i.e., neither inactivated nor spontaneously clotted).

For example, ready-to-use bovine fibrinogen stored (without protease inhibitors) at room temperature (˜23° C.) is optimally maintained at pH 6.5 to 9.0, preferably at approximately pH 6.5 to 8.2, to retain the ability to rapidly form a clot when the stored preparation is neutralized and exposed to thrombin/Ca⁺⁺. When ready-to-use bovine fibrinogen (without protease inhibitors) is stored under refrigeration (˜4° C.), the optimal pH is also optimally maintained at pH 6.5 to 9.0, preferably at approximately pH 6.5 to 8.2, more preferably at pH 6.5 to 7.07 to retain the ability to rapidly form a clot when the stored preparation is neutralized and exposed to thrombin/Ca⁺⁺ (see Table 2).

The pH of the storage-stable fibrinogen solution is determined by the buffer in which it is stored. For example, in the Examples that follow, solutions of bovine fibrinogen (50 mg protein/mL) were freshly prepared in one of the following 0.1 M buffers: histidine, pH 7.24; glycine pH 9.31; or carbonate, pH 9.05 or pH 9.86.

In a preferred embodiment of the invention the storage-stable bovine fibrinogen solution is prepared in histidine buffer, although other recognized, physiologically acceptable buffers known to the art may be used to prepare the storage-stable fibrinogen, so long as the resulting pH of the fibrinogen solution remains within the proscribed range, such that it's activity is maintained, but it remains without spontaneous clotting.

Currently available, commercial fibrinogen contains salts used in the isolation and purification process. As noted in the Examples, this includes sodium citrate and sodium chloride, but the presence of such salts that are a residual part of the fibrinogen purification process do not appear to affect the storage-stability of the resulting preparation. Since the purpose of the present invention is to produce a storage-stable, ready-to-use, fibrinogen solution that will retain the characteristics of a comparable, freshly prepared fibrinogen solution, the effect of the fibrinogen purification process would be the same for both. Nevertheless, the high concentrations of citrate and/or sodium may affect clotting of the stored fibrinogen preparation. The present method is, therefore, effective, even if the identified salts or other chelators are present in the freshly prepared solution, and the storage stable preparation will retain the characteristics and activity of a comparable freshly-prepared solution, so long as activity is maintained during storage and spontaneous clotting is not induced by the salt or chelator.

For the purposes of the Examples that follow, sodium azide (0.025%) was added to each sample as an antimicrobial agent. Although the antimicrobial agent may have, to some extent, induced spontaneous clotting, it does not appear to have had such an effect. In a preferred embodiment of the present invention, no antimicrobial agent is added, and sterility is preserved using known techniques. However, in an alternative embodiment, antimicrobial agents are added to the extent exemplified, to avoid microbial contamination of the fibrinogen solution over long term storage. Any recognized, physiologically antimicrobial agent is acceptable for the purposes of the present invention, so long as the activity of the fibrinogen solution is maintained throughout the length of the storage and spontaneous clotting is not induced.

The storage-stable fibrinogen solution of the present invention may be supplemented with, and act as a carrier vehicle for: growth factor(s), drug or other compond(s) or mixtures thereof, so long as noted above, the activity of the fibrinogen solution is maintained throughout the length of the storage and spontaneous clotting is not induced. For example, by supplementing the fibrinogen preparation with a growth factor, the ready-to-use fibrinogen when used to prepare a fibrin sealant or tissue adhesive preparation can accelerate, promote or improve wound healing, tissue (re)generation. Such a supplemented preparation may also comprise additional components, e.g., drug(s), antibody(ies), anticoagulant(s) and other compounds that: (1) potentiate, stimulate or mediate the biological activity of the growth factor(s) or other additive(s) or component(s); (2) decrease the activities of one or more additive(s) or component(s) of the growth-factor supplemented fibrinogen or fibrin sealant or tissue adhesive prepared therefrom, wherein such activities would inhibit or destroy the growth factor(s) in the preparation; (3) allow prolonged delivery of the additive or component from a preparation, such as a fibrin sealant or tissue adhesive, made from the ready-to-use fibrinogen solution of the present invention; and (4) possess other desirable properties. The contemplated additive(s) or supplement(s) are intended to also include any mutants, derivatives, truncated or other modified forms thereof, which possess similar biological activity(ies), or a subset thereof, to those of the compound or composition from which it is derived.

More than one additive or component may be simultaneously added to or supplied by the storage-stable fibrinogen solution of the present invention. Although the concentration of such additive(s) and/or component(s) will vary in the fibrinogen solution depending on the objective, the concentration must be sufficient to allow such compound(s) and/or composition(s) to accomplish their intended or stated purpose. The amount of such supplement(s) to be added can be empirically determined by one of ordinary skill in the art by testing various concentrations and selecting that which is effective for the intended purpose and site of application. Dyes, tracers, markers and the like may also be added, for example, to examine the subsequent delivery of the material to which the fibrinogen is added.

In a preferred embodiment of the invention, protease inhibitors (PI), such as, but not limited to aprotinin (e.g., 5 μg/ml final concentration) or PPACK (e.g., 25 μM final concentration) are added in an effective amount to the stored, aqueous fibrinogen solution. Other protease inhibitors (PI) are known in the art and may be substituted for the aprotinin and PPACK disclosed in Example 2. Notably, aprotinin is used in the commercially available Tisseal product. By an “effective amount” of a protease inhibitor is meant that amount of PI that will prevent proteolysis of the fibrinogen sample. This amount would vary based upon the PI or combination of Pis used, but could be readily determined by one of ordinary skill in the art. However, although the stored fibrinogen solution may remain stable for a longer period of time in the presence of a PI, it is known that PI effects decay with time.

For example, although the addition of a PI to the storage-stable bovine fibrinogen preparation prevented undesirable, spontaneous clot formation in the long-term storage of the protein at ˜4° C., the addition of PI does not appear to be effective for use in producing a rapid fibrinogen/thrombin product (fibrin clot) at, for example, 149 days. However, rapid fibrinogen/thrombin clot formation was seen in storage-stable, bovine fibrinogen solution samples maintained at room temperature (˜23° C.) at pH 6.3 to 7.07 for at least 149 days.

As shown in Tables 2 and 3, “inhibition” equates to “prevention,” i.e., the PIs are initially active under the presently disclosed conditions (that is, clotting is inhibited/prevented), but then the activity of the PI declines, after which the inhibiting effect diminishes and eventually ceases. The rate of decline of PI activity in the fibrinogen solution is pH and temperature dependent.

The Examples accompanying the present disclosure indicate, by continuous observation and testing, that the fibrinogen solutions of the invention under the preferred conditions remain stable (active and not spontaneously clotted) for at least 97 days at pH 6.5 to 9.0, when stored at room temperature (˜23° C.), and for at least 149 days at pH 6.5 to 8.1 in the presence of a protease inhibitor, when stored at ˜4° C., but for only 7 days in the absence of the PI. Thus, the fibrinogen solutions of the preferred embodiments of the invention comprising fibrinogen plus PI, remain stable for years at room temperature, and for months absent the PI.

In light of proven stability of the bovine fibrinogen solution, the product is shown to be stable for extremely long periods of time, as compared with known deep frozen or lyophilized preparations of the concentrated protein that have been maintained without a substantial loss of activity (i.e., fibrinogen/thrombin fibrin clots are still rapidly formed upon mixing), even years after the initial storage of the fibrinogen product. Thus, “long term storage” means storage of the fibrinogen solution in ready-to-use form under the presently disclosed conditions, without substantial loss of protein activity for at least 3 days, preferably for at least 3 weeks, more preferably for at least 13 weeks, even more preferably for at least 149 days, even more preferably for at least 1 year, and most preferably for a period greater than 1 year. In addition, the term is meant to further include a period of frozen storage in addition to storage in the ready-to-use form, which would add additional years to the storage of the product.

The present invention relates to any fibrinogen preparation, but the methods of the present invention are directed to the stable storage of ready-to-use, aqueous fibrinogen solutions from any mammalian species. Although bovine fibrinogen is described by example, the invention is not intended to be so limiting. There appears to be no species compatibility issues associated with the use of the stored fibrinogen with other mammalian species. For example, the subject bovine fibrinogen may be used following storage in aqueous solution to prepare, e.g., a biologically compatible tissue adhesive preparation for use in or on any species of mammal.

As a blood plasma protein, fibrinogen is frequently accompanied by a risk of contamination with blood-borne pathogens, such as those possibly contaminating plasma proteins, in particular, hepatitis viruses or HIV. Therefore, one skilled in the art would readily prepare fibrinogen so as to remove potentially infectious materials. Common techniques to achieve this goal include, but are not limited to, ultrafiltration, pasturization (heating), solvent detergent treatment, radiation exposure and ultraviolet light treatment. Although virus inactivation by high heating or steam methods are impractical for biologically active protein solutions, including the present fibrinogen solutions, nanofiltration is an optional treatment for the fibrinogen solution of the present invention before placing it into the sterile storage container.

Nevertheless, although fibrinogen is unstable to heat and thus inactivated during the conventional liquid heating process, processes have been developed for heating fibrinogen to inactivate any potentially contaminating viruses, e.g., hepatitis or HIV, without inactivating the fibrinogen per se. U.S. Pat. No. 5,116,950 (Miyano et al., issued May 26, 1992) provides a process for heating fibrinogen which comprises heating an aqueous solution containing fibrinogen in the presence of at least a sugar, an amino acid and a magnesium salt until the virus(es) possibly contaminating said fibrinogen are inactivated.

In a preferred embodiment of the invention, the aqueous solution of fibrinogen, thus heated, may be further purified, if desired, and processed in a conventional manner such as by dialysis, sterilization or filtration. Also, various washing steps can be employed to remove stabilizing additives by methods known in the art.

The fibrinogen solutions of the present invention are ideally suited for forming a physiological fibrin structure when exposed to an activator solution, and fibrin clots are rapidly formed. This is proven by mixing the stored fibrinogen solution with an equal volume of a thrombin/CaCl₂ solution (comprising, e.g., 2.5 units/mg fibrinogen (100 units/ml) thrombin and 3-6 mM excess CaCl₂ over citrate or other chelators that may be added to solutions), as set forth in the Examples which follow. If the resulting clot demonstrates a physiological fibrin structure, it will have the typical, spatial branched fibril structure shown when clots are formed by the action of thrombin on freshly-prepared or freshly isolated and purified bovine fibrinogen under physiological conditions, i. e., at an ionic strength of approximately 0.15 and approximately neutral pH.

Fibrinogen and thrombin concentrations dictate time to clot formation, clot strength, clot adhesion, and thus hemostasis.

Moreover, the fibrinogen preparation and/or the fibrinogen-based tissue adhesive to which it is added according to the present invention has no cytotoxic effect when used as a tissue adhesive, i.e., it is “biocompatible,” meaning that it is well tolerated by cells, permits a good cell growth and offers an ideal prerequisite for good wound healing therewith. This is proven by dilution of the tissue adhesive with the equal volume of the half-isotonic or isotonic sodium chloride solution, and addition to fibroblast growth media. No damaging effect on the fibroblasts is detectable (See Redl et al., 1985).

Thus, the present storage-stable, ready-to-use fibrinogen solutions are prepared in a manner which meets all of the demands of a tissue adhesive, namely biocompatibility, viral safety and high adhesive strength, plus it has the advantage of simple and rapid preparation from a ready-to-use fibrinogen product. The tissue adhesive prepared from the storage stable fibrinogen of the present invention may be thus used in any known manner in which such biologically-prepared, supplemented or unsupplemented tissue adhesives are used, e.g., pharmacologic or cosmetic uses, including for infusion purposes, such as delivery of antibiotics, antineoplastics, anesthetics, and the like; for wound healing, coagulation, and fibrinogenaemia; for inhibition of operative or post-operative sequelae; for coating prostheses; for dressable wound sealings and for safe and sustained hemostasis, namely sealing fluid and/or air leakage, and the like in a patient.

The invention is further described by example. The examples, however, are provided for purposes of illustration to those skilled in the art, and are not intended to be limiting. Moreover, the examples are not to be construed as limiting the scope of the appended claims. Thus, the invention should in no way be construed as being limited to the following examples, but rather, should be construed to encompass any and all variations which become evident as a result of the teaching provided herein.

### EXAMPLES

To evaluate the storage-stability of the fibrinogen solutions of the present invention, the stability, solubility and clotting activity of fibrinogen solutions were assessed over a range storage conditions having different buffers (pH values), temperatures, and additives such as protease inhibitors. Bovine fibrinogen, bovine thrombin, aprotinin, buffer solutions, calcium chloride, sodium hydroxide and hydrochloric acid were purchased from Sigma Chemical Company, St. Louis, Mo. PPACK was supplied by Calbiochem, San Diego, Calf. Bovine fibrinogen was certified to contain 61% protein (97% clottable) and 39% salts.

Standard research grade fibrinogen contains salts used in the isolation and purification process. This includes sodium citrate and sodium chloride. Thus, a 40 mg/ml solution of fibrinogen, contains, for example, 54 mM sodium citrate and 419 mM sodium chloride in addition to the fibrinogen. Additionally, sodium azide (0.025%) was added to each sample as an antimicrobial agent.

The clotting assays were completed in the following manner in general accordance with Kasper, Proc. Symposium on Recent Advances in Hemophilia Care, Los Angeles, Calif. Apr. 13-15, 1989 (in Liss, N.Y., 1990). Aliquots (100 μl) of each fibrinogen sample were added to 4 ml polypropylene test tubes. Each sample was neutralized (pH 7.0-7.3) using 0.1 M sodium hydroxide, 0.2 M histidine buffer (pH 6.0) or 0.1 M hydrochloric acid (determined in preliminary studies using larger volumes)). Thrombin was prepared as 200 units/ml with 1 M calcium chloride (3-6 mM excess of calcium over sodium citrate in fibrinogen preparations). The thrombin preparation was then diluted with 0.1 M histidine buffer (pH 7.2) to a final thrombin concentration of 100 units/ml (2.5 units of thrombin per mg of fibrinogen). All samples were assayed at room temperature (23±2° C.).

Clotting was measured by timing the reaction that occurred when 100 □1 of thrombin was added to the fibrinogen sample (100 □1), and the mixture was vigorously mixed. Times were recorded when the solution turned to a viscous gel (a drastic slowing of the liquid being mixed) and to a solid clot (the point at which all liquid ceased movement upon mixing). The time to solid clot formation was often twice the time of gel formation.

### Example 1

Stability of Aqueous Bovine Fibrinogen Stored at Room Temperature, pH 7-10.

To evaluate the ability to store the fibrinogen solutions of the invention for long periods of time at room temperature, the stability, solubility and clotting activity of a fibrinogen solution were evaluated following storage for at least 149 days (21 weeks) at a constant temperature of 20-25° C. Solutions of bovine fibrinogen (50 mg protein/mL) were freshly prepared on day 1 of the storage period in one of the following 0.1 M buffers: histidine, pH 7.24; glycine pH 9.31; or carbonate, pH 9.05 or pH 9.86.

The solutions were inspected for clarity and spontaneous clotting. A manual clotting assay was performed at 25° C. by neutralizing the solutions to pH 7.0-7.5, and adding thrombin (125 units/mg fibrinogen), and 3-5 mM excess CaCl₂ over citrate in the fibrinogen solution. The preparation was mixed vigorously, and the time required for a clot to form was measured as described above, and recorded.

Clotting results of bovine fibrinogen in histidine buffer at pH 7.24, stored at room temperature (˜23° C.) are shown in Table 1. In all samples, from day 1 through day 149, the fibrinogen solutions remained clear and unclotted until thrombin was added.

TABLE 1

Clotting time (in seconds)

| Day | PH 7.24 | pH 9.05 | pH 9.31 | pH 9.86 |
|---|---|---|---|---|
| 1 | NT | NT | NT | NT |
| 3 | 9 | 8 | 8 | 27 |
| 36 | 10 | >300 | >300 | >300 |
| 72 | 9.5 | >300 | >300 | >300 |
| 149 | >300 | NT | NT | NT |

NT= not tested.

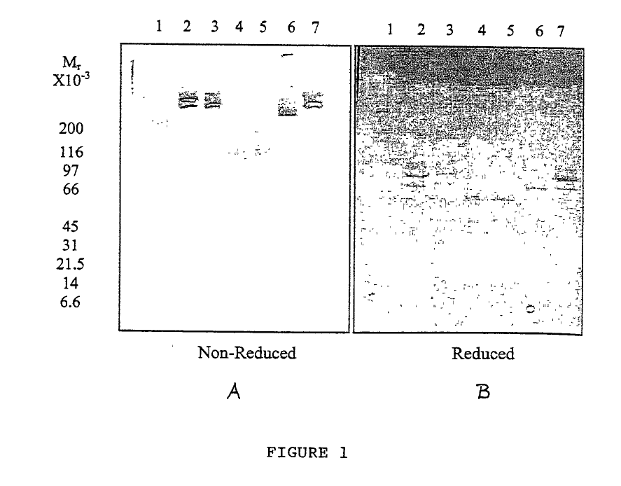

The protein integrity of the fibrinogen formulations were assessed by sodium dodecyl sulphate (SDS)-polyacrylamide gel electrophoresis (SDS-PAGE) on day 44. (Standard SDS PAGE conditions are described, e.g., Laemmli, Nature 227:680-685 (1970)). The SDS-PAGE analysis showed that the samples of bovine fibrinogen that had been stored at pH 7.24 (FIG. 1, lane 3) migrated at essentially the same rate as the freshly prepared bovine fibrinogen (BFG) control (FIG. 1, lanes 2 and 7) in non-reduced and reduced gels. By comparison, the samples stored at higher pH (shown in FIG. 1, lanes 4, 5 and 6), appeared degraded and/or aggregated. The degradation was greatest at pH 9.05-9.31 (FIG. 1, lanes 4 and 5), with less degradation and more aggregation (or clotting) in the pH 9.86 sample.

Focusing on the bovine fibrinogen solution at pH 7.24, the sample remained clear and unclotted at day 149 of storage at room temperature. However, in a single clotting assay, the pH 7.24 sample did not clot upon addition of thrombin. The pH of the pH 7.24 sample was determined to be 6.98 following the addition of thrombin. Neutral pH is optimal for thrombin. Nevertheless, the sample appeared to have lost the ability to clot between day 73 and day 147.

It was concluded, therefore, that bovine fibrinogen, prepared as an aqueous solution in histidine buffer at pH 7.24, was stable to storage at room temperature for more than 10 weeks. However, it appeared unable to clot at 21 weeks.

### Example 2

Stability of Aqueous Fibrinogen Solutions Stored at Two Temperatures, with and without Protease Inhibitors

To further evaluate the ability to store aqueous fibrinogen solutions for long periods of time, the stability, solubility and clotting activity of both bovine fibrinogen solutions were evaluated following storage for at least 149 days (over 21 weeks) over a range of five pH values (pH 6.50 to pH 9.87), with and without protease inhibitors (PI), at room temperature (˜23° C.) and refrigerated (4° C.). Duplicate solutions of bovine fibrinogen (39 mg protein/ml) were freshly prepared on day 1 of the storage period in one of the following 0.1 M buffers: histidine, pH 6.0 or 7.2; Tris pH 8.16; glycine pH 9.3; or carbonate, pH 9.1 or pH 9.9. Protease inhibitors: PPACK (25 μM final concentration) and aprotinin (5 μg/mL final concentration) were added to one-half of the duplicates before storage.

To evaluate clotting ability, samples were neutralized according to the previously-redetermined protocol, and then tested for clotting as described in the stability study in Example 1.

Clotting results are shown for bovine fibrinogen in Table 2 at the conditions shown.

TABLE 2
Clotting times for bovine fibrinogen, stored at 23° C. and 4° C.,
no protease inhibitors.

| Age in | Temp. in | Clotting Time (in seconds) | | | | |
|---|---|---|---|---|---|---|
| Days | ° C. | pH 6.5 | pH 7.36 | pH 8.2 | pH 9.04 | pH 9.87 |
| 4 | 23 | 12 | 13 | 15 | 12 | 210 |
| | 4 | 10 | 9 | 15 | 10 | Clotted |
| 7 | 23 | 10 | 10 | 11 | 11 | 240 |
| | 4 | 11 | 10 | 10 | 10 | Clotted |
| 22 | 23 | 9 | 10 | 10 | >300 | >300 |
| | 4 | Partial | Partial | Clotted | Clotted | Clotted |
| | | clot | clot | | | |
| 97 | 23 | 10 | 100 | >300 | >300 | Clotted |
| | 4 | Clotted | Clotted | Clotted | Clotted | Clotted |
| 149 | 23 | Clotted | >300 | >300 | >300 | >300 |
| | 4 | Clotted | Clotted | Clotted | Clotted | Clotted |

Note: “Clotted” refers to spontaneous clotting, absent addition of thrombin.

By day 22, the bovine samples at 4° C. had all spontaneously clotted. By comparison, when examined at day 97, the samples of bovine fibrinogen stored at room temperature were mostly clear, except at the highest pH.

TABLE 3
Clotting times for bovine fibrinogen with protease inhibitors,
stored at 23° C. and 4° C.

| Age in | Temp. in | Clotting Time (in seconds) | | | | |
|---|---|---|---|---|---|---|
| Days | ° C. | pH 6.31 | pH 7.07 | pH 8.10 | pH 9.09 | pH 9.80 |
| 4 | 23 | 40 | 30 | 120 | 26 | 300 |
| | 4 | >300 | >300 | >300 | 180 | 60 |
| 7 | 23 | 15 | 25 | 60 | 20 | >300 |
| | 4 | >300 | >300 | 40 | 60 | 22 |
| 22 | 23 | 15 | 12 | 20 | 65 | >300 |
| | 4 | >300 | 100 | 95 | 15 | 15 |
| 97 | 23 | 30 | 28 | >300 | >300 | >300 |
| | 4 | 18 | 24.5 | 21 | NT | 130 |
| 149 | 23 | 180 | 125 | >300 | >300 | >300 |
| | 4 | 25 | 15 | 15 | Clotted | >300 |

NT = not tested.
“Clotted” refers to spontaneous clotting, absent addition of thrombin.

Samples containing PI (PPACK or aprotinin) evaluated after storage at ˜° C. or ˜4° C. residual PH-dependent results. The diminished ability to clot appears to have been due to the residual ability of the PI in the fibrinogen solution to inhibit the added thrombin. Therefore, Shorter term storage at ˜4° C. (4-22 days) resulted in the effective inhibition of thrombin-dependent Clotting, i.e., samples did not clot after thrombin was added because thrombin activity was inhibited by residual PI inhibitors remaining in solution

However, because PI components decay with time, their activity declines accordingly. After a longer period of storage (22-149 days), PI activity had decayed, thereby allowing the addition of thrombin to trigger clotting of the fibrinogen sample. Again, the reactions were pH-depend

As a result, it was concluded that following storage for at least 149 days, the best conditions for storing bovine fibrinogen in aqueous solution is at a pH ranging from 6.31 to 7.07 at room temperature, or at 40° C. at a pH ranging from pH 6.31 to pH 8.10 in the presence of a protease inhibitor.

Each and every patent, patent application and publication that is cited in the foregoing specification is herein incorporated by reference in its entirety.

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the spirit and scope of the invention. Such modifications, equivalent variations and additional embodiments are also intended to fall within the scope of the appended claims.

What is claimed is:

1. A storage-stable, concentrated, ready-to-use, biocompatible mammalian fibrinogen solution, wherein stability of the fibrinogen solution is pH and temperature dependent.

2. The fibrinogen solution of claim 1, wherein the fibrinogen is fully solubilized, and wherein the solution is aqueous.

3. The fibrinogen solution of claim 2, wherein stability is maintained for a storage period ranging from at least one (1) day to one or more years following initial preparation.

4. The fibrinogen solution of claim 3, wherein the fibrinogen solution comprises a pH-controlling buffer selected from the group consisting of histidine, Tris, glycine or carbonate.

5. The fibrinogen solution of claim 4, wherein the solution is buffered to a pH ranging from pH 6.5 to 8.2 and the storage temperature is maintained under refrigeration at a temperature of about 4° C.

6. The fibrinogen solution of claim 5, wherein storage buffer is histidine.

7. The fibrinogen solution of claim 5, wherein stability is maintained for at least about 10 weeks.

8. The fibrinogen solution of claim 4, wherein the solution is buffered to a pH ranging from pH 6.5 to 8.2 and the storage temperature is maintained at room temperature.

9. The fibrinogen solution of claim 8, wherein stability is maintained for at least 7 days.

10. The fibrinogen solution of claim 8, wherein stability is maintained for at least 22 days.

11. The fibrinogen solution of claim 4, wherein the solution is buffered to a pH ranging from pH 6.31 to 8.1, the storage temperature is maintained at a temperature ranging from about 40° C. to about 23° C., and an effective amount of a protease inhibitor is added to the fibrinogen solution prior to storage to prevent proteolysis of the fibrinogen sample.

12. The fibrinogen solution of claim 11, wherein stability is maintained for at least 7 days.

13. The fibrinogen solution of claim 11, wherein storage stability is maintained for at least 22 days.

14. The fibrinogen solution of claim 11, wherein stability is maintained for at least 97 days.

15. The fibrinogen solution of claim 11, wherein stability is maintained for at least 149 days.

16. The fibrinogen solution of claim 3, wherein the mammalian fibrinogen is bovine.

17. A method of stably storing mammalian fibrinogen in a ready-to-use, aqueous solution, comprising:
preparing a freshly prepared fibrinogen solution or freshly isolating and purifying fibrinogen solution from plasma or from a frozen fibrinogen preparation under sterile conditions; and
storing the fibrinogen solution at refrigeration temperature,
wherein the fibrinogen solution remains liquid, and at pH levels ranging from pH 6.5 to 8.2, and under conditions wherein biocompatibility and biological activity of the fibrinogen is maintained.

18. The fibrinogen solution of claim 17, further comprising maintaining stability for a storage period ranging from at least one (1) day to one or more years following initial preparation.

19. The method of claim 18, wherein the refrigeration temperature is maintained at about 40° C.

20. The method of claim 18, wherein stability is maintained for at least 7 days.

21. A method of stably storing mammalian fibrinogen in a ready-to-use, aqueous solution, comprising:
storing a freshly prepared or freshly isolated and purified fibrinogen solution or one prepared from a frozen fibrinogen preparation under sterile conditions, and
maintaining the stored fibrinogen solution at room temperature temperature,
wherein the fibrinogen solution remains liquid, and at pH levels ranging from pH 6.5 to 8.2, and under conditions wherein biocompatibility and biological activity of the fibrinogen is maintained.

22. The fibrinogen solution of claim 21, further comprising maintaining stability for a storage period ranging from at least one (1) day to one or more years following initial preparation.

23. The method of claim 22, wherein stability is maintained for at least 7 days.

24. The method of claim 22, wherein stability is maintained for at least 22 days.

25. A method of stably storing mammalian fibrinogen in a ready-to-use, aqueous solution, comprising:
freshly preparing a fibrinogen solution, or freshly isolating and purifying a fibrinogen solution from plasma or from a frozen fibrinogen preparation under sterile conditions;
adding to the fibrinogen solution an effective amount of a protease inhibitor to prevent proteolysis of the fibrinogen sample; and
storing the fibrinogen solution at (i) a constant temperature ranging from about 4° C. to about 23° C., wherein the fibrinogen solution remains liquid; (ii) at pH levels ranging from pH 6.31 to 8.1, (iii) under conditions wherein biocompatibility and biological activity of the fibrinogen is maintained.

26. The method of claim 25, further comprising maintaining stability for a storage period ranging from at least one (1) day to one or more years following initial preparation.

27. The method of claim 26, wherein stability is maintained for at least 7 days.

28. The method of claim 26, wherein stability is maintained for at least 22 days.

29. The method of claim 26, wherein stability is maintained for at least 97 days.

30. The method of claim 26, wherein stability is maintained for at least 149 days.
